(19) United States
(12) Patent Application Publication
(10) Pub. No.: US 2025/0297985 A1
(43) Pub. Date: Sep. 25, 2025

(54) PORE CHIP CASE

(71) Applicant: ADVANTEST CORPORATION, Tokyo (JP)

(72) Inventors: Yasuharu IMAI, Tokyo (JP); Kosuke OINUMA, Tokyo (JP); Nobuei WASHIZU, Tokyo (JP)

(21) Appl. No.: 19/229,087

(22) Filed: Jun. 5, 2025

Related U.S. Application Data
(63) Continuation of application No. PCT/JP2023/043688, filed on Dec. 6, 2023.

(30) Foreign Application Priority Data
Dec. 6, 2022 (JP) 2022-195238

(51) Int. Cl. G01N 27/447

(52) U.S. Cl. CPC G01N 27/44791

(57) ABSTRACT

A pore chip case houses a pore chip. A main body includes a chip housing space, a first chamber, and a second chamber. The pore chip is housed in the chip housing space and is supported in a perpendicular plane. The first chamber and the second chamber adjoin in the horizontal direction and are partitioned by the pore chip.

### CROSS REFERENCE TO RELATED APPLICATIONS

This application is a continuation under 35 U.S.C. § 120 of PCT/JP2023/043688, filed Dec. 6, 2023, which is incorporated herein by reference, and which claimed priority to Japanese Application No. 2022-195238, filed Dec. 6, 2022. The present application likewise claims priority under 35 U.S.C. § 119 to Japanese Application No. 2022-195238, filed Dec. 6, 2022, the entire content of which is also incorporated herein by reference.

### BACKGROUND

### 1. Technical Field

The present disclosure relates to measurement with use of a pore device.

### 2. Description of the Related Art

Method for measuring particle size distribution called electrical sensing zone method (based on the Coulter's principle) has been known. In this measurement method, an electrolyte solution that contains particles is allowed to pass through a pore called nanopore. During passage of each particle through the pore, the electrolyte solution in the pore will decrease the volume by an amount equivalent to the volume of the particle, thus increasing electric resistance of the pore. The volume (or, particle size) of the particle can therefore be determined, by measuring the electric resistance of the pore.

FIG. 1 is a block diagram illustrating a microparticle measurement system 1R making use of the electrical sensing zone method. A microparticle measurement system 1R has a pore device 100, a measuring instrument 200R, and a data processor 300.

The inside of the pore device 100 is filled with an electrolyte solution 2 that contains particles 4 to be detected. The inside of the pore device 100 is partitioned by a pore chip 102 into two chambers, in which an electrode 106 and an electrode 108 are individually provided. Under potential difference generated between the electrode 106 and the electrode 108, an ion current flows between the electrodes, during which the particles 4 migrate from one chamber through the pore 104 into the other chamber while driven by electrophoresis.

The measuring instrument 200R generates a potential difference between the pair of electrodes 106, 108, and acquires information correlated with resistivity Rp between the electrode pair. The measuring instrument 200R has a transimpedance amplifier 210, a voltage source 220, and a digitizer 230. The voltage source 220 is structured to generate a potential difference Vb between the pair of electrodes 106, 108. The potential difference Vb provides a driving force of electrophoresis, as well as a bias signal for measuring the resistivity Rp.

Between the pair of electrodes 106, 108, there flows microcurrent Is which is inversely proportional to the resistivity of the pore 104.

$$Is = Vb / Rp \qquad ( 1 )$$

The transimpedance amplifier 210 is structured to convert the microcurrent Is into a voltage signal Vs. Given a conversion gain as r, an equation below holds.

$$Vs = - r \times Is \qquad ( 2 )$$

Substitution of equation (1) into the equation (2) gives equation (3) below.

$$Vs = - Vb \times r / Rp \qquad ( 3 )$$

The digitizer 230 is structured to convert the voltage signal Vs into digital data Ds. In this way, the voltage signal Vs inversely proportional to the resistivity Rp of the pore 104 is obtainable, with use of the measuring instrument 200R.

FIG. 2 is an exemplary waveform chart of the microcurrent Is measured by the measuring instrument 200R. Note that the ordinates and abscissae of a waveform chart and a time chart referred to herein are appropriately enlarged or shrunk for easy understanding, and also the waveforms illustrated herein are simplified, exaggerated or emphasized for easy understanding.

During a short period of passage of each particle, the resistivity Rp of the pore 104 increases. The current Is therefore decreases in a pulsated manner; every time one particle passes. Amplitude of each pulse current correlates with the particle size. The data processor 300 is structured to process the digital data Ds, and to analyze count, particle size or the like of the particles 4 contained in the electrolyte solution 2.

FIG. 3 is a cross-sectional view of a pore device examined by the present inventors. A pore device 100R has a pore chip 110 and a pore chip case 800R. The pore chip 110 has a pore 112.

The pore chip case 800R is structured to support the pore chip 110 in a horizontal plane. The pore chip case 800R has, inside thereof, a first chamber 802 and a second chamber 804 partitioned by the pore chip 110. The first chamber 802 communicates with the outside through a flow path 806, meanwhile the second chamber 804 communicates with the outside through a flow path 808.

Prior to the measurement, a solution is injected through the flow path 806 into the first chamber 802, meanwhile the solution is injected through the flow path 808 into the second chamber 804.

In a package having the pore chip 110 arranged horizontally as illustrated in FIG. 3, an axis of opening of the pore is aligned to the vertical direction. Therefore, upon introduction of the solution into the chambers 802, 804, the pore 112 will tend to have an air pocket 8 formed therein, since the pore 112 is eventually sandwiched by the solution from above and below.

Formation of the air pocket 8 in the pore 112 will block the solution from entering the pore 112, whereby the current will not be able to flow through the pore 112. Also, the particle will not be able to pass through the pore. In is not easy to remove the air pocket 8 once generated. Ultrasonic cleaning, for example, would be a possible method for removing the air pocket. The method is, however, considered to be less reliable, and the ultrasonic vibration would damage a membrane in which the pore is formed.

A possible method for suppressing the air pocket from generating would be subjecting the pore chip typically to plasma treatment, so as to impart hydrophilicity. The treatment is, however, time-consuming and would push up the cost.

### SUMMARY

The present disclosure has been arrived at in consideration of such circumstances, and one exemplary embodiment thereof is to provide a pore chip case capable of suppressing the air pocket from generating.

One embodiment of the present disclosure relates to a pore chip case structured to house a pore chip. A main body of the pore chip case is structured to support the pore chip in a perpendicular plane. The main body has a first chamber and a second chamber which adjoin in the horizontal direction and partitioned by the pore chip.

Note that also free combinations of these constituents, and also any of the constituents and expressions exchanged among the method, apparatus, and system, are valid as the modes of this invention.

### BRIEF DESCRIPTION OF DRAWINGS

FIG. 1 is a block diagram illustrating a microparticle measurement system making use of the electrical sensing zone method;

FIG. 2 is an exemplary waveform chart of microcurrent Is measured with a measuring instrument;

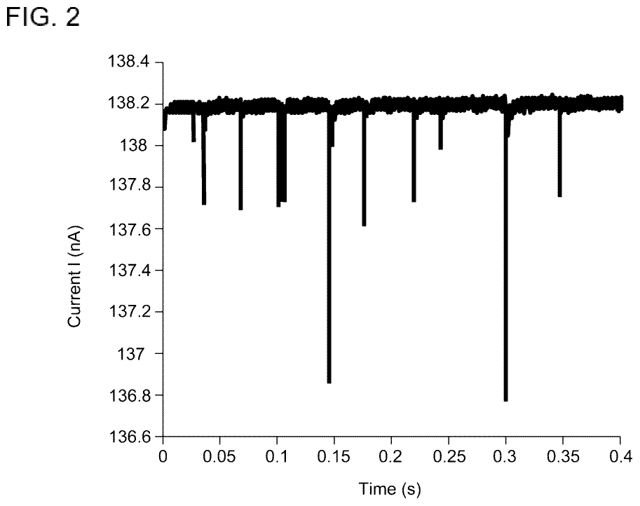

FIG. 3 is a cross-sectional view of a pore device examined by the present inventors;

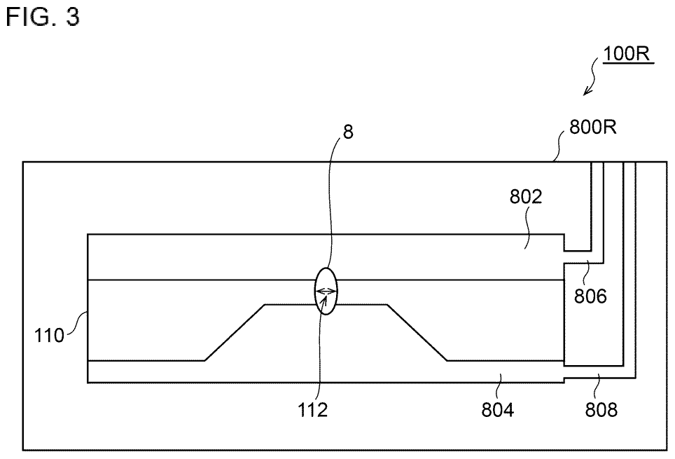

FIG. 4 is a perspective view illustrating a pore chip case according to one embodiment;

FIG. 5 is a cross-sectional view illustrating the pore chip case;

FIG. 6 is a cross-sectional view illustrating how the pore chip case houses the pore chip;

FIG. 7 is a cross-sectional view illustrating the pore chip case specifically around the pore chip; and

FIG. 8 is a diagram illustrating relationships between water pressure p and count of passed particle.

### DESCRIPTION OF EMBODIMENTS

### Outline of Embodiments

Some exemplary embodiments of the present disclosure will be outlined. This outline is intended for briefing some concepts of one or more embodiments, for the purpose of basic understanding of the embodiments, as an introduction before detailed description that follows, without limiting the scope of the invention or disclosure. This outline is not an extensive overview of all possible embodiments and is therefore intended neither to specify key elements of all embodiments, nor to delineate the scope of some or all of the embodiments. For convenience, the term “one embodiment” may be used to designate a single embodiment (Example or Modified Example), or a plurality of embodiments (Examples or Modified Examples) disclosed in the present specification.

A pore chip case according to one embodiment is structured to house a pore chip. The pore chip case has a main body structured to support the pore chip in a perpendicular plane. The main body has a first chamber and a second chamber which are adjoined in the horizontal direction and partitioned by the pore chip.

In this structure, a solution filled in the first chamber and the second chamber will elevate its liquid level, from bottom to top. This successfully suppresses an air pocket from generating in the pore.

In one embodiment with the pore chip case filled with a liquid, the level of height of a liquid face of the liquid may be higher by 10 mm or more, than a level of height of the pore.

This structure can afford elevation of the water pressure at the pore as compared with the prior art and can therefore improve transmittance of the particle and measurement efficiency.

In one embodiment, the main body may have: a first flow path that extends from a level lower than a pore level of the pore chip in the first chamber, towards a top face of the main body; a second flow path that extends from a level higher than the pore level in the first chamber, towards the top face of the main body; a third flow path that extends from a level lower than the pore level in the second chamber, towards the top face of the main body; and a fourth flow path that extends from a level higher than the pore level in the second chamber, towards the top face of the main body.

In one embodiment, the level of height of the top face of the main body may be higher by 10 mm or more, than the level of height of the pore of the pore chip.

In one embodiment, the main body may further have a protrusion which is formed on the top face of the main body, so as to separate the first flow path and the second flow path, from the third flow path and the fourth flow path. This successfully prevents short-circuiting between the solution in the first chamber and the solution in the second chamber.

In one embodiment, the pore case may further have a first electrode provided on a wall face of the first flow path, and a second electrode provided on a wall face of the third flow path.

In one embodiment, the pore case may further have an electrode sheet connected to a bottom face of the main body. The first electrode and the second electrode may be formed on the electrode sheet.

In one embodiment, the main body may be dividable into a first part and a second part, with the perpendicular plane as a boundary.

In one embodiment, a pore device having the pore chip, and the pore chip case structured to house the pore chip, and a measuring instrument having an interface socket to which the pore device is attached may be included.

### EMBODIMENT

A preferred embodiment will be explained below, referring to the attached drawings. All similar or equivalent constituents, members and processes illustrated in the individual drawings will be given same reference numerals, so as to properly avoid redundant explanations. The embodiment is merely illustrative and is not restrictive about the invention. All features and combinations thereof described in the embodiment are not always necessarily essential to the present invention.

In the present specification, a “state in which a member A is coupled to a member B” includes a case where the member A and the member B are physically and directly coupled, and a case where the member A and the member B are indirectly coupled while placing in between some other member that does not substantially affect the electrically coupled state, or does not degrade the function or effect demonstrated by the coupling thereof.

Similarly, a “state in which member C is provided between member A and member B” includes a case where the member A and the member C, or the member B and the member C are directly connected, and a case where they are indirectly connected, while placing in between some other member that does not substantially affect the electrical connection state among the members, or does not degrade the function or effect demonstrated by the members.

Dimensions (thickness, length, width, etc.) of the individual members illustrated in the drawings may be appropriately enlarged or shrunk for easy understanding. Furthermore, the dimensions of the plurality of members do not necessarily indicate the dimensional relationship among them, so that a certain member A, if depicted thicker than another member B in a drawing, may even be thinner than the member B.

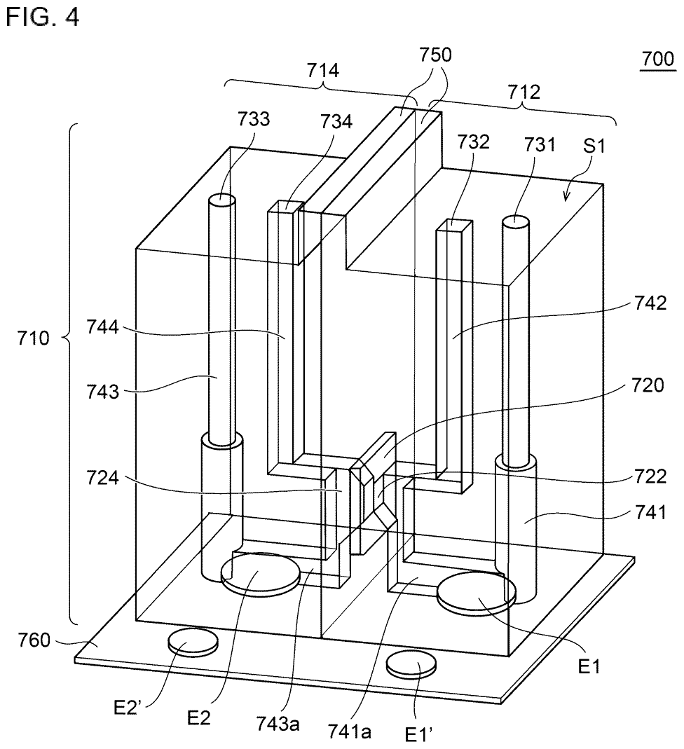

FIG. 4 is a perspective view illustrating a pore chip case 700 according to one embodiment. The pore chip case 700 has a main body 710 and an electrode sheet 760. The main body 710 is structured to house and support a pore chip, in a chip housing space 720 provided therein.

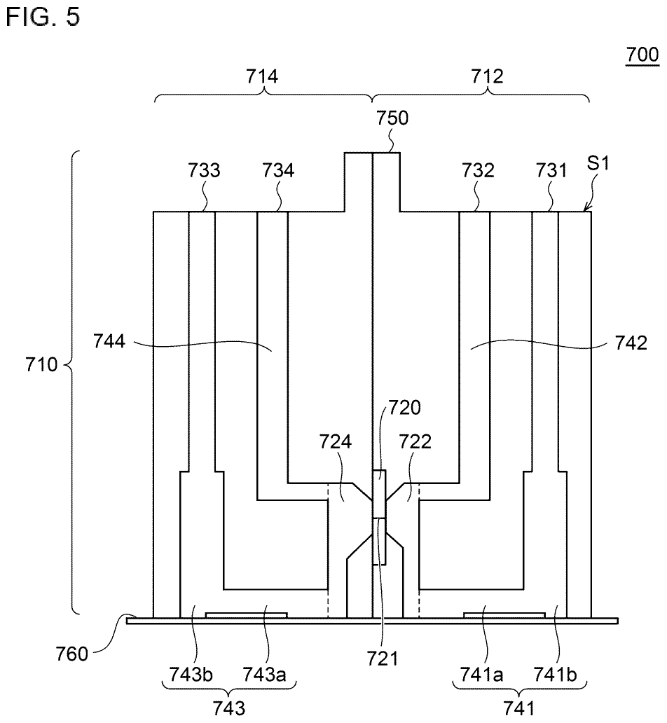

FIG. 5 is a cross-sectional view of the pore chip case 700. The main body 710 has a first chamber 722, the chip housing space 720, and a second chamber 724 which adjoin in the horizontal direction. With the pore chip housed in the chip housing space 720, the first chamber 722 and the second chamber 724 are partitioned by the pore chip.

Inside the main body 710, there are formed a first flow path 741 and a second flow path 742 which extend from the first chamber 722 towards a top face S1 of the main body 710. More specifically, the first flow path 741 extends from a level lower than a pore level 721 of the pore chip in the first chamber 722, towards a first opening 431 of the top face S1. The second flow path 742 extends from a level higher than the pore level 721 in the first chamber 722, towards a second opening 732 of the top face S1. The first flow path 741 is L-shaped and has horizontally laid part 741a and vertically laid part 741b. The second flow path 742 is also L-shaped.

Similarly, inside the main body 710, there are formed a third flow path 743 and a fourth flow path 744 which extend from the second chamber 724 towards the top face S1 of the main body 710. More specifically, the third flow path 743 extends from a level lower than the pore level 721 of the pore chip in the second chamber 724, towards a third opening 433 of the top face S1. The fourth flow path 744 extends from a level higher than the pore level 721 in the second chamber 724, toward a fourth opening 734 of the top face S1. Also, the third flow path 743 is L-shaped and has horizontally laid part 743a and vertically laid part 743b. The fourth flow path 744 is also L-shaped.

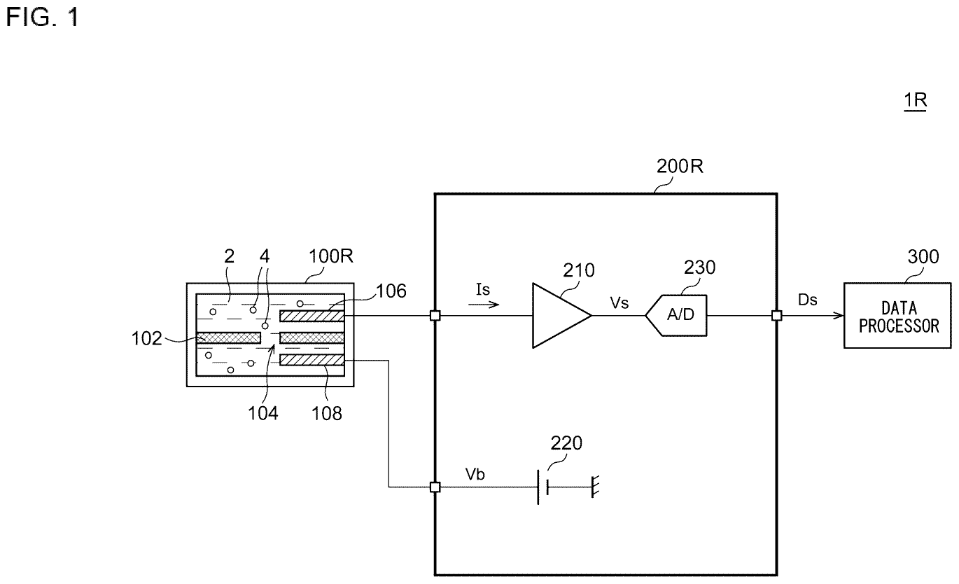

The main body 710 has a first electrode E1 and a second electrode E2. The first electrode E1 is provided in the first chamber 722 or in the first flow path 741. The second electrode E2 is provided in the second chamber 724 or in the second flow path 742. The first electrode E1 and the second electrode E2 correspond to the electrodes 106, 108 in FIG. 1, respectively.

Referring now back to FIG. 4. The electrode sheet 760 is attached to a bottom face of the main body 710. The electrode sheet 760 also serves as an inner wall of a horizontally laid part 741a of the first flow path 741, and a horizontally laid part 743a of the third flow path 743. The electrode sheet 760 has the first electrode E1 formed in a part that corresponds to the inner wall of the first flow path 741, and the second electrode E2 formed in a part that corresponds to the inner wall of the third flow path 743.

The electrode sheet 760 has a contact electrode Ec1 electrically connected to the first electrode E1, and a contact electrode Ec2 electrically connected to the second electrode E2. At the time of measurement, a voltage signal is applied to the contact electrodes Ec1 and Ec2.

The main body 710 is dividable into a first part 712 and a second part 714. The chip housing space 720 is formed in the first part 712.

On the top face S1 of the main body 710, and at a boundary between the first part 712 and the second part 714, formed is a protrusion 750 by which the first flow path 741 and the second flow path 742 are separated from the third flow path 743 and the fourth flow path 744. The protrusion 750 can prevent short-circuiting between the solution in the first chamber 722 and the solution in the second chamber 424.

With the pore chip case filled with the liquid, the level of height of the liquid face of the liquid may be preferably higher by 10 mm or more, than the pore level 721. The level of height of the liquid face may be considered as the level of height of the top face S1.

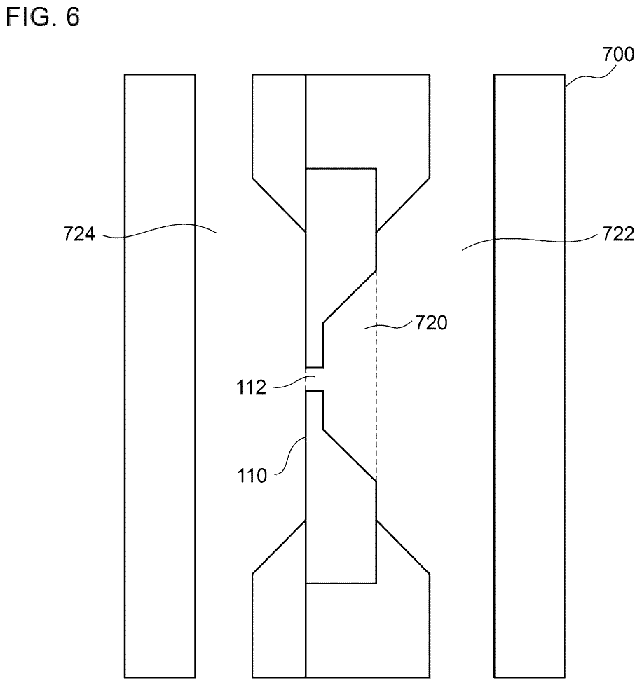

FIG. 6 is a cross-sectional view illustrating how the pore chip case 700 houses the pore chip 110. The pore chip 110 has a pore 112. The pore chip 110 is housed in the chip housing space 720 of the pore chip case 700, while being kept upright in the vertical direction.

The structure of the pore chip case 700 has been described. Next, the advantage will be described.

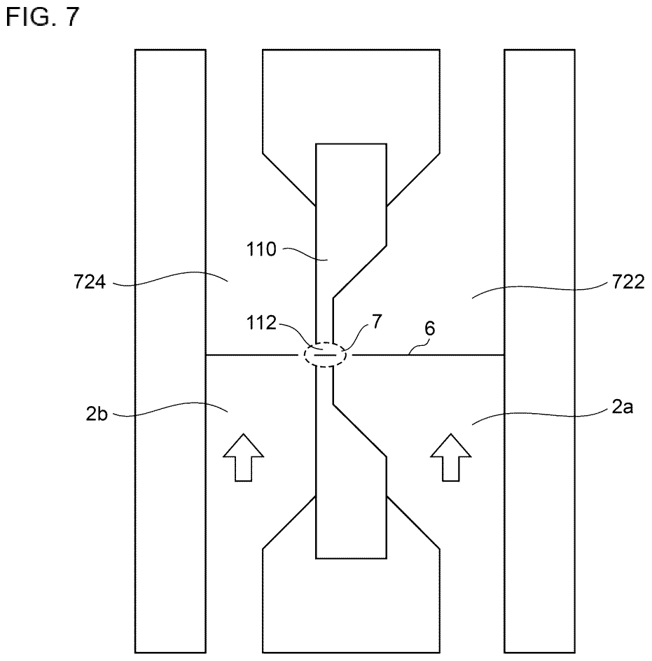

FIG. 7 is a cross-sectional view illustrating the pore chip case 700 specifically around the pore chip 110. In a preparatory phase of measurement, an electrolyte solution 2 is injected through the first opening 731. The electrolyte solution 2a flows through the first flow path 741 to enter the first chamber 722. Similarly, an electrolyte solution 2b is injected through the third opening 733. The electrolyte solution 2b flows through the third flow path 743 to enter the second chamber 724. In the first chamber 722 and the second chamber 724, a liquid face 6 of the electrolyte solutions 2a, 2b elevate from bottom to top. Air 7 having resided in the pore 112 of the pore chip 110 can be released upwards, as the liquid face 6 elevates. This successfully suppresses the air pocket from generating.

The pore chip case 700 also has the following advantages. The pore chip case 700 has a level difference of 10 mm or larger, between the pore 112 and the level of height of the top face S1. This means that water pressure in the pore 112 increases. The water pressure p is determined by the height h measured from the pore up to the water surface, which is given by the equation below:

p=ρ·gh,

where, ρ is density of water, and g is gravitational acceleration.

Electrolyte solution often used for example in microparticle detection system is a chloride solution (NaCl, KCl, PBS, etc.) at a concentration of lower than 1 mol. The density of the solution may therefore be assumed almost same as that of water, without significant influence. Given

ρ=997kg/m³

and

g=9.81m/s²,

the water pressure p will be 9781×h [kPa]. Given h=10 mm=0.01 m, then p=0.09781 kPa will be obtained.

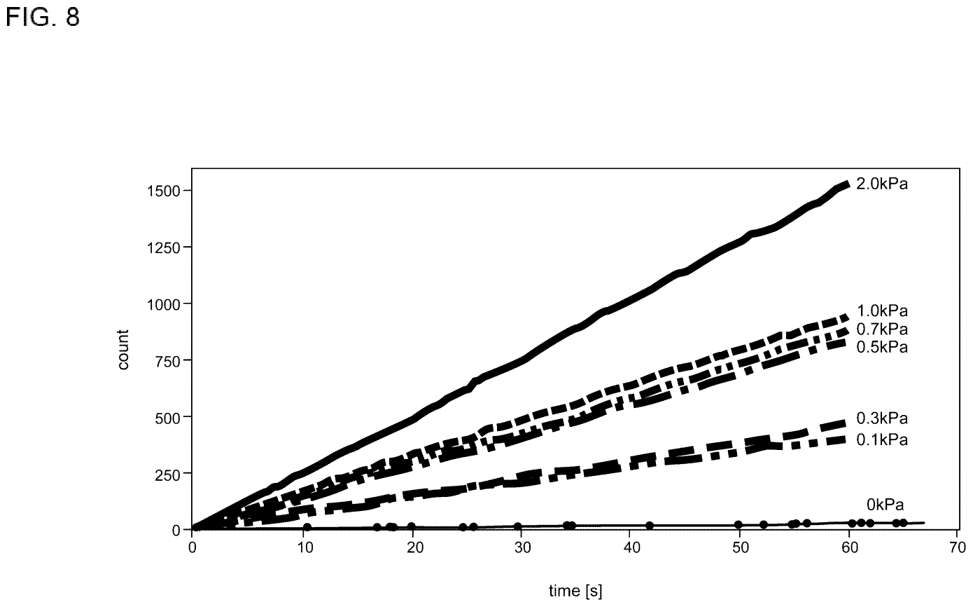

FIG. 8 is a diagram illustrating relationships between the water pressure p and count of passed particle. The abscissa plots time, and the ordinate plots the count of passed particle. The result teaches that the count of passed particle per unit time (1 minute) increases as the water pressure p increases. Given h=10 mm and p≈0.1 kPa, it follows that 6.6 or more particles are detectable per minute. Considering that approximately 200 particles in total are necessary for a sufficient level of measurement accuracy, this embodiment can complete the measurement within 30 minutes.

The prior pore chip could achieve approximately 3 particles/min as the count of passed particle. This means that the measurement takes one hour. Use of the pore chip case 700 of this embodiment can almost halve the measurement time.

The embodiment has been described. It is to be understood by those skilled in the art that the embodiment is merely illustrative, that the individual constituents or combinations of various processes may be modified in various ways, and that also such modifications fall within the scope of the present disclosure. Such modified examples will be explained below.

The electrodes E1 and E2, having been formed on the electrode sheet 760 in the embodiment, are not limited regarding the position and shape. For example, the electrode E1 may be formed in the first chamber 722 or in the second flow path 742. Similarly, the electrode E2 may be formed in the second chamber 724 or in the fourth flow path 744.

The electrodes E1 and E2 may alternatively be rod-shaped probe electrodes. The electrode E1 in this mode may be inserted through the first opening 731 or the second opening 732, or may be inserted through another opening provided on the side face of the first part 712, so that at least a part of the probe electrode is exposed to any one of the first flow path 741, the first chamber 722, or the second flow path 742. The same applies to the electrode E2.

Having described herein the microparticle measurement device, the present invention is not limited thereto and is widely applicable to measuring instruments responsible for microcurrent measurement with use of the pore device, such as a DNA sequencer.

Having described the present invention with reference to the embodiment, the embodiment merely illustrates the principle and applications of the present invention, allowing a variety of modifications and layout change without departing from the spirit of the present invention specified by the claims.

What is claimed is:

1. A pore chip case for housing a pore chip, comprising:
a main body structured to support the pore chip in a perpendicular plane, and having a first chamber and a second chamber which adjoin in a horizontal direction and are partitioned by the pore chip.

2. The pore chip case according to claim 1, wherein, upon being filled with a liquid inside of the pore chip case, a level of height of a liquid face of the liquid is higher by 10 mm or more, than a level of height of a pore of the pore chip.

3. The pore chip case according to claim 1, wherein the main body comprises:
a first flow path that extends from a level lower than a pore level of the pore chip in the first chamber, towards a top face of the main body;
a second flow path that extends from a level higher than the pore level in the first chamber, towards the top face of the main body;
a third flow path that extends from a level lower than the pore level in the second chamber, towards the top face of the main body; and
a fourth flow path that extends from a level higher than the pore level in the second chamber, towards the top face of the main body.

4. The pore chip case according to claim 3, wherein a level of height of the top face of the main body is higher by 10 mm or more, than the level of height of the pore of the pore chip.

5. The pore chip case according to claim 3, wherein the main body further comprises a protrusion structured to separate the first flow path and the second flow path, from the third flow path and the fourth flow path, on the top face of the main body.

6. The pore chip case according to claim 3, further comprising:
a first electrode provided on a wall face of the first flow path; and
a second electrode provided on a wall face of the third flow path.

7. The pore chip case according to claim 6, further comprising an electrode sheet connected to a bottom face of the main body, and
the first electrode and the second electrode are formed on the electrode sheet.

8. The pore chip case according to claim 1, wherein the main body is dividable into a first part and a second part, with the perpendicular plane as a boundary.

9. A microparticle measurement system comprising:
a pore device having a pore chip, and the pore chip case according to claim 1 structured to house the pore chip; and
a measuring instrument having an interface socket to which the pore device is attached.
